(19) United States
(12) Patent Application Publication
(10) Pub. No.: US 2002/0182272 A1
(43) Pub. Date: Dec. 5, 2002

(54) Methods of treatment of HIV-associated conditions

(76) Inventor: Bruce Halstead, Grand Terrace, CA (US)

Correspondence Address: Robert D. Fish, Rutan & Tucker, LLP, PO Box 1950, Costa Mesa, CA, 92626-1950, US

(21) Appl. No.: 10/159,433

(22) Filed: May 29, 2002

Related U.S. Application Data
(60) Provisional application No. 60/294,479, filed on May 30, 2001.

(51) Int. Cl. A61K 35/78; A61K 31/195

(52) U.S. Cl. 424/725; 514/564

(57) ABSTRACT

A method of treating an HIV-related condition includes a step in which a patient is diagnosed as being infected with an HIV virus and having an HIV related condition. In a further step, a composition that comprises at least one of a chelator and an antiviral agent is administered to the patient, wherein the antiviral agent comprises a plant extract or a synthetic or isolated compound from a plant that is demonstrated to have an antiviral effect.

This application claims the benefit of U.S. provisional application No. 60/294,479 filed May 30, 2001, incorporated herein by reference in its entirety.

### FIELD OF THE INVENTION

The field of the invention is treatment of HIV-associated conditions.

### BACKGROUND OF THE INVENTION

Most patients infected with the HIV virus will develop AIDS, reflecting a breakdown in their immune system's capability to ward off foreign and “self”-generated antigens. For example, Kaposi sarcoma, and numerous bacterial and yeast infections are relatively common diseases associated with AIDS. Typically, these secondary diseases are treated with drugs that specifically target the etiologic agent (e.g., sarcoma cell, bacterium, or virus) of those diseases, thereby often increasing an already long list of undesired side effects brought on by attempts to control the propagation of the HIV virus.

### DETAILED DESCRIPTION

The inventors contemplate that treatment of HIV related conditions can be significantly improved by administration of a composition that comprises at least one of a chelator and an antiviral agent, wherein the antiviral agent comprises a plant extract, or a synthetic or isolated compound from a plant that is demonstrated to have an antiviral effect.

The term “HIV related condition” as used herein refers to intrinsic and extrinsic challenges to an immune system that may develop into an apparent (i.e., detectable by diagnotic tools) disease while the patient has a detectable HIV serum virus titer. Particularly contemplated conditions include bacterial infections (e.g., pneumocystis carnii, tuberculosis, salmonellosis, mycobacterium avium complex, etc.), viral infections (e.g., cytomegalovirus, herpes simplex, hepatitis, varicella zoster, Epstein-barr, etc.), fungal infections (e.g., candidiasis, cryptococcal meningitis, histoplasmosis, etc.), parasite infections (e.g., toxoplasmosis, cryptosporidiosis, etc.), and Kaposi sarcoma.

Suitable compositions are described in copending provisional patent applications with the title “Treatment of Virus Using Chelator and Antiviral Agent” by Bruce Halstead et al., filed on or about May 30, 2001, “Time Release Chelators” by Bruce Halstead et al., filed on or about May 30, 2001, and “Time Release reverse transcriptase inhibitors” by Bruce Halstead et al., filed on or about May 30, 2001, all of which are incorporated by reference herein.

In a preferred aspect of the inventive subject matter, a method of treating a patient comprises one step in which a patient infected with HIV is diagnosed with an HIV related condition. In a further step, a composition is administered to the patient that comprises at least one of a chelator and an antiviral agent, wherein the antiviral agent comprises a plant extract or a synthetic or isolated compound from a plant that is demonstrated to have an antiviral effect. It should be recognized that all patients infected with an HIV virus may be treated using contemplated methods, however, patients with a CD4⁺ count of less than 200 are particularly contemplated. Consequently, especially preferred patients include patients with developing or fully developed AIDS, wherein such patients may or may not receive pharmacological treatment.

With respect to the administration of contemplated compounds, it should be appreciated that a particular dosage and regimen will typically depend on the particular HIV-related condition. It is generally contemplated that the dosage, route and formulation is substantially identical or similar to those described in the copending provisional applications. However, where appropriate, alternative dosages, routes, and formulations may be employed, and in fact all dosages formulations and routes are contemplated that result in a positive response of the patient to the administration.

Thus, specific embodiments and applications of treatment of HIV-related conditions have been disclosed. It should be apparent, however, to those skilled in the art that many more modifications besides those already described are possible without departing from the inventive concepts herein. The inventive subject matter, therefore, is not to be restricted except in the spirit of the appended contemplated claims. Moreover, in interpreting both the specification and the contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A method of treating an HIV-related condition, comprising:
diagnosing a patient that is infected with an HIV virus with an HIV related condition; and
administering to the patient a composition that comprises at least one of a chelator and an antiviral agent, wherein the antiviral agent comprises a plant extract or a synthetic or isolated compound from a plant that is demonstrated to have an antiviral effect.

2. The method of claim 1 wherein the HIV-related condition is a viral infection.

3. The method of claim 1 wherein the HIV-related condition is a bacterial infection.

4. The method of claim 1 wherein the HIV-related condition is a parasite infection.

5. The method of claim 1 wherein the HIV-related condition is a Kaposi sarcoma.

6. The method of claim 1 wherein the chelator chelates at least one of Ca²⁺ and Mg²⁺.

7. The method of claim 6 wherein the chelator is selected from the group consisting of 1,2Bis(2-aminophenoxy)ethane-N,N,N′,N′-tetra-acetic acid, Ethylenebis(oxyethylenenitrilo)tetra-acetic acid, 1,2-bis(2-aminophenoxy)ethane-N,N,N′,N′-tetraacetic acid tetrakis(acetoxymethyl ester), trans-1,2-diaminocyclohexane-tetraacetic acid, and diethyllenetriamine-pentaacetic acid.

8. The method of claim 6 wherein the chelator is selected from the group consisting of tri-methylaminetricarboxylic acid, poly(aspartic acid), and poly(glutamic acid).

9. The method of claim 6 wherein the chelator is ethylenediamine-N,N,N′,N′-tetraacetic acid.

10. The method of claim 1 wherein the plant extract is prepared from a plant selected from the group consisting of Abies webbiana, Acacia spec. Acacia Arabia, Agrimonia eupatoria, Ajuga decumbens, Allium cepa, Allium sativum, Aloe vera, Alternanthera philoxeroides or sessiles, Ammi maius, Andographis paniculata, Apium graveolens, Apium leptophyllum, Arachis hypogaea, Arctium lappa, Amebia euhcroma, Asparagus racemosus, Astragalus spinosus, Astragalus lentingosis swainsonine, Buchenavia capita, Bryonia cretica ssp. Dioica, Bryonia angustifolia, Camellia theifera, Camellia sinensis, Cedrela toona, Chrysanthemum morifolium, Coffea arabica, Coptis chinesis, Coptis teetoides, Coptis japonica, Coraria nepalensis, Coriandrum sativum, Curcuma longa, Datura metel syn alba, Daucus carota, Echinacea angustiflora and purpurea, Echinacea simulata, Echinacea pallida, Epimedium grandiflorum, Epimedium sagittatum, Epimedium sinense, Epilobium angustifolium, Erigeron Canadensis, Eugenia or Syzigium claviflorum, Fagara xanthox, Foeniculum vulgarel, Gardenia coronaria, Gaultheria trichophylla, Glycine max, Glycyrrhiza labra, Gossypium herbaceum, Heracleum sphondylium, Hypericum perforatum, Hypericum japonicum, Hyssopus officinalis, Jasminum officinale, Lithospermum erythrorhizon, Lonicera japonica, Luffa luffa, Lycopus europaeus, Magnolia officinalis, Mallotus repandus, Mallotus philippinesis, Matricaria chamomil, Matricaria recutitia, Melissa parviflora, Melissa officinalis, Momordica balsamina, Momordica charantia, Narcissus tazetta, Narcissus pseudonarcissus, Oenthera rosea, Paeonia spec., Papaver somniferum, Perilla frutescens, Phyllanthus niruri, Pinus koraicenis, Pinus parviflora, Piper nirgum, Plumeria rubra, Polyantha suberosa, Prunella vulgaris, Prunus bakariensis, Prunus amygdalus, Psoralea corylifolia, Randia dunatorum, Raphanus sativus, Rheum palmatum, Rhus coriaria, Rhus chinesis, Ricinus communis, Rosmarinus officinalis, Salvia miltiorhiza and officinalis, Sambucus ebulus, Saussurea lappa, Scilla griffithii, Scutellaria baicalensis baiealein, Sedum sediforme, Senecio scandens, Senecio aereus, Skimmia laureola, Solarium niporum, Swertia franchetiana, Terminalia chebula, Terminalia catappa, Terminalia alata, Thula occidentalis, Trapalaponica spec., Trichosanthes dioica, Trichosanthes kirilowii, Urtica dioica, Viola yeodensis, Woodfordia fruticosa, Woodwardia spec., and Zanoxylum nitidum.

11. The method of claim 1 wherein the synthetic compound synthesized de novo.
